(19) United States
(12) Patent Application Publication
(10) Pub. No.: US 2023/0117284 A1
(43) Pub. Date: Apr. 20, 2023

(54) PROCESS FOR THE SYNTHESIS OF S-BEFLUBUTAMID FROM (R)-2-AMINOBUTANOIC ACID

(71) Applicant: Cheminova A/S, Philadelphia, PA (US)

(72) Inventors: Ravindra V. DATAR, Bangalore (IN); Jianhua MAO, Shanghai (CN)

(21) Appl. No.: 17/798,871

(22) PCT Filed: Feb. 10, 2021
(86) PCT No.: PCT/IB2021/000075

§ 371 Date: Aug. 10, 2022

Related U.S. Application Data
(60) Provisional application No. 62/972,802, filed on Feb. 11, 2020.

(51) Int. Cl. C07C 231/02; C07C 51/363

(52) U.S. Cl. CPC C07C 231/02; C07C 51/363

(57) ABSTRACT

Disclosed are methods for preparing compound S-1 (S-1) from (R)-2-bromobutanoic acid prepared by treating (R)-2-aminobutanoic acid with an alkali metal nitrite compound and hydrobromic acid.

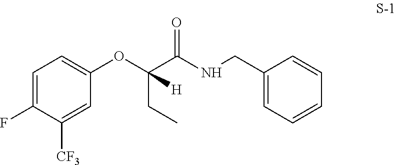 [SMILES: CC[C@H](Oc1ccc(F)c(C(F)(F)F)c1)C(=O)NCc1ccccc1]

### FIELD OF THE INVENTION

This invention relates to a method for preparing the (S)-enantiomer of beflubutamid.

### BACKGROUND OF THE INVENTION

U.S. Pat. No. 4,929,273 discloses N-benzyl-2-(4-fluoro-3-trifluoromethylphenoxy)-butanoic amide of Formula 1 as an herbicidal compound. It has a single asymmetric center at the 2-carbon of the amide moiety and thus can be a chiral molecule.

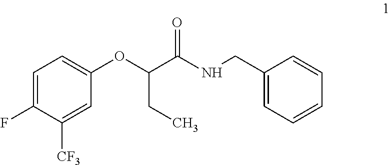 [SMILES: CCC(Oc1ccc(F)c(C(F)(F)F)c1)C(=O)NCc1ccccc1]

This compound in racemic form has been marketed commercially under the common name beflubutamid as a soil herbicide for pre- and post-emergence control of dicotyledonous weeds in cereals. It inhibits the enzyme phytoene-desaturase that is involved in the biosynthesis of carotenoids. Depletion of carotenoids leads to photooxidation of chlorophyll and bleaching/chlorosis of susceptible weeds.

U.S. Pat. No. 4,929,273 also discloses that the (−)-optical isomer is more herbicidally active than the racemic mixture. The more active enantiomer has been identified as having the (S)-configuration shown as compound S-1 (Environ. Sci. Technol. 2013, 47, 6806-6811 and Environ. Sci. Technol. 2013, 47, 6812-6818).

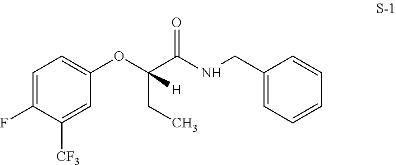 [SMILES: CC[C@H](Oc1ccc(F)c(C(F)(F)F)c1)C(=O)NCc1ccccc1]

While the methods disclosed in the preceding reference can provide the desired compound S-1, continuous improvements are sought, particularly in the development of methods to provide materials on a commercial scale. Therefore, the need continues for new methods that are less costly, more efficient, more flexible, or more convenient to operate.

### SUMMARY OF THE INVENTION

Embodiment A. This invention provides a method for preparing compound S-1

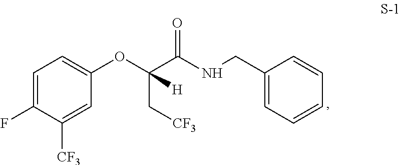 [SMILES: O=C(NCc1ccccc1)[C@H](CC(F)(F)F)Oc1ccc(F)c(C(F)(F)F)c1]

from compound R-2

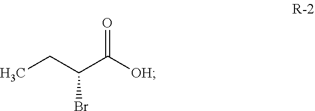

wherein compound R-2 is prepared by

treating compound R-3

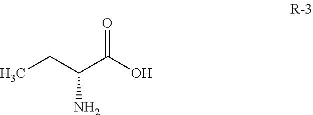

with an alkali metal nitrite and hydrobromic acid.

Embodiment B. This invention also provides a method for preparing compound S-1

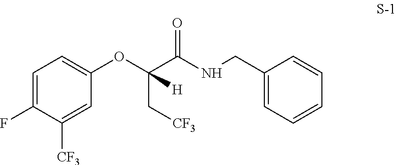 [SMILES: O=C(NCc1ccccc1)[C@H](CC(F)(F)F)Oc1ccc(F)c(C(F)(F)F)c1]

from compound R-2

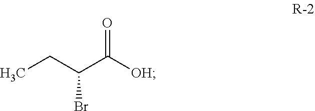

wherein compound R-2 is prepared by

treating compound R-3

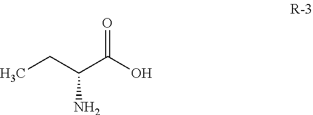

with an alkali metal nitrite and hydrobromic acid;
the method further comprising converting compound R-2 to compound S-1.

Embodiment C. This invention also provides a method for preparing compound S-1

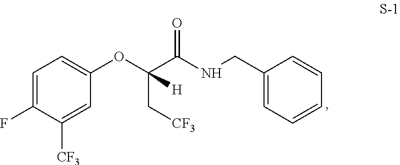 [SMILES: O=C(NCc1ccccc1)[C@H](CC(F)(F)F)Oc1ccc(F)c(C(F)(F)F)c1]

the method comprising:

treating compound R-3

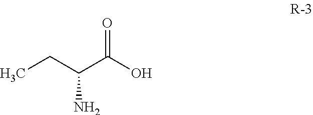

with an alkali metal nitrite and hydrobromic acid to prepare compound R-2

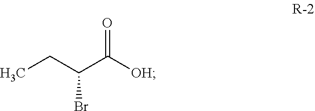

and

converting compound R-2 to compound S-1.

Embodiment D. This invention also provides a method for preparing compound S-1

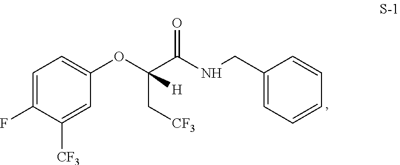 [SMILES: O=C(NCc1ccccc1)[C@H](CC(F)(F)F)Oc1ccc(F)c(C(F)(F)F)c1]

the method comprising:

treating compound R-3

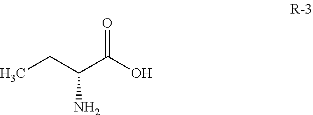

with an alkali metal nitrite and hydrobromic acid to prepare compound R-2

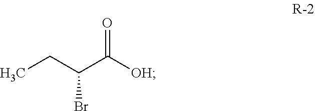

treating compound R-2 with a chlorinating agent to prepare compound R-8

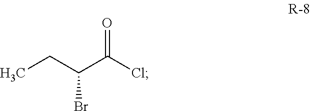

treating compound R-8 with compound 7 (i.e. benzylamine)

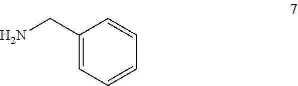

to prepare compound R-9

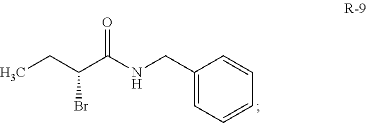

and

treating compound R-9 with compound 5 (i.e. 4-fluoro-3-(trifluoromethyl)phenol)

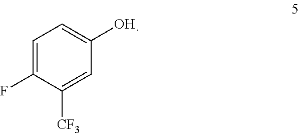 [SMILES: Oc1ccc(F)c(C(F)(F)F)c1]

### DETAILED DESCRIPTION OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “suitable” indicates that the entity or condition so described is appropriate for use in the situation or circumstance indicated. As used herein, the terms “treatment” or treating” denotes using a chemical or chemical process to alter the existing condition of other materials, chemicals or compounds. The term “converting” refers to causing an entity such as a chemical compound to change in structure, form, character or function. For example, a compound of a first formula or structure is converted to a compound of a second formula or structure by a chemical process involving one or more treatments as defined above.

In the above recitations, the term “alkyl” or “alkane”, used either alone or in compound words such as “haloalkane” includes straight-chain or branched alkyl, such as methyl, ethyl, n-propyl, i-propyl, or the different butyl, pentyl or hexyl isomers. “Alkoxy” includes, for example, methoxy, ethoxy, n-propyloxy, isopropyloxy and the different butoxy, pentoxy and hexyloxy isomers. “Alkanol” indicates an alkane alcohol including, for example, methanol, ethanol, n-propanol, isopropanol and the different butanol, pentanol and hexanol isomers.

As used herein, “alkali metal” refers to elements of group 1 of the periodic table, including lithium, sodium, potassium and cesium, preferably sodium or potassium, or cations thereof, such as when used in combination with an anionic counterion to define a chemical compound.

The term “halogen”, either alone or in compound words such as “halogenase” includes fluorine, chlorine, bromine or iodine. The term “chlorinating agent” refers to a reagent that introduces a chlorine atom into a chemical compound.

The total number of carbon atoms in a substituent group is indicated by the “Cᵢ-Cⱼ” prefix where i and j are numbers from 1 to 6.

The term “optionally” when used herein means that the optional condition may or may not be present. For example, when a reaction is conducted optionally in the presence of a solvent, the solvent may or may not be present.

This invention includes compounds that are enantiomerically enriched compared to the racemic mixture; for example, in an enantiomer of the compound of Formula S-1 or any intermediate in a process described herein for preparing the compound of Formula S-1. Also included are the essentially pure enantiomers of compounds of Formula S-1 or any intermediate in a process described herein for preparing the compound of Formula S-1.

When enantiomerically enriched, one enantiomer is present in greater amounts than the other, and the extent of enrichment can be defined by an expression of enantiomeric excess (“ee”), which is defined as (Fₘₐⱼ−Fₘᵢₙ)·100%, where Fₘₐⱼ is the mole fraction of the dominant enantiomer in the mixture and Fₘᵢₙ is the mole fraction of the lesser enantiomer in the mixture (e.g., an ee of 20% corresponds to a 60:40 ratio of enantiomers).

As used herein, compounds having at least an 80% enantiomeric excess; preferably at least a 90% enantiomeric excess; more preferably at least a 94% enantiomeric excess, at least a 96% enantiomeric excess, or at least a 98% enantiomeric excess of a specific isomer are designated as R- or S-, depending on the predominant configuration at the asymmetric center. Of note are essentially enantiomerically pure embodiments (>99% ee) of the more predominant enantiomer. As used herein, compounds having less than 80% enantiomeric excess are designated as scalemic.

Molecular depictions drawn herein generally follow standard conventions for depicting stereochemistry. To indicate stereoconfiguration, bonds rising from the plane of the drawing and towards the viewer are denoted by solid wedges where the broad end of the wedge is attached to the atom rising from the plane of the drawing towards the viewer as shown below, where group B is rising from above the plane of the drawing. Except where specifically indicated, hydrogen atoms attached to the asymmetric center are generally not shown.

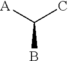

Bonds going below the plane of the drawing and away from the viewer are denoted by dashed wedges where the broad end of the wedge is attached to the atom further away from the viewer, i.e. group B′ is below the plane of the drawing.

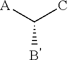

Constant width lines indicate bonds with a direction opposite or neutral relative to bonds shown with solid or dashed wedges; constant width lines also depict bonds in molecules or parts of molecules in which no stereoconfiguration is intended to be specified. Notably as used herein, a constant width line attached to an asymmetric center also represents a condition where the amounts of R- and S-configuration at that center are equal; e.g., a compound with a single asymmetric center is racemic. When a racemic mixture is intended for any specific compound herein, it will be denoted with the prefix “rac-”

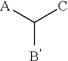

Racemic Mixture or “rac”

Embodiments of the invention include the following.

Embodiment A1. The method of Embodiment A wherein compound R-2 is converted to compound S-1 by the method comprising

treating compound R-2 with a C₁-C₆ alkanol to prepare a compound of Formula R-4;

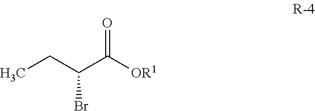

wherein R¹ is C₁-C₆ alkyl;

treating a compound of Formula R-4 with compound 5

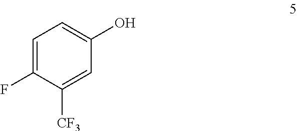 [SMILES: Oc1ccc(F)c(C(F)(F)F)c1]

to prepare a compound of Formula S-6

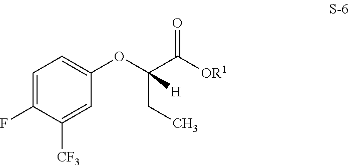

wherein R¹ is C₁-C₆ alkyl; and

treating a compound of Formula S-6 with compound 7

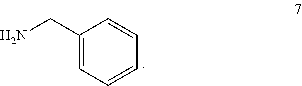

Embodiment A2. The method of Embodiment A1 wherein treating compound R-2 to prepare the compound of Formula R-4 comprises

treating compound R-2 with a chlorinating agent to prepare compound R-8

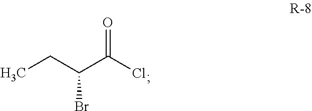

and

treating compound R-8 with a C₁-C₆ alkanol or a salt thereof.

Embodiment A3. The method of Embodiment A2 wherein the chlorinating agent is thionyl chloride.

Embodiment A4. The method of any of Embodiments A1 through A3 wherein R¹ is CH₃.

Embodiment A5. The method of Embodiment A wherein compound R-2 is converted to compound S-1 by the method comprising

treating compound R-2 with a chlorinating agent to prepare compound R-8

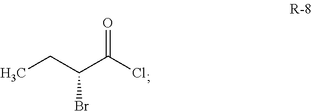

treating compound R-8 with compound 7

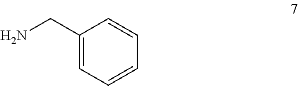

to prepare compound R-9

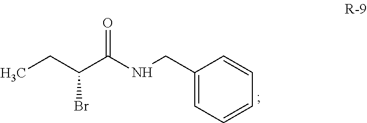

and

treating compound R-9 with compound 5

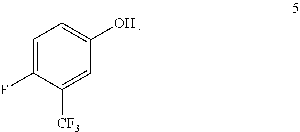 [SMILES: Oc1ccc(F)c(C(F)(F)F)c1]

Embodiment A6. The method of Embodiment A5 wherein the chlorinating agent is thionyl chloride.

Embodiment B1. The method of Embodiment B wherein compound R-2 is converted to compound S-1 by the method comprising

treating compound R-2 with a C₁-C₆ alkanol to prepare a compound of Formula R-4;

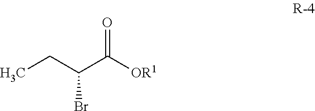

wherein R¹ is C₁-C₆ alkyl;

treating a compound of Formula R-4 with compound 5

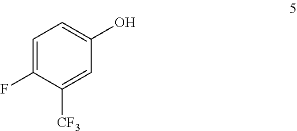 [SMILES: Oc1ccc(F)c(C(F)(F)F)c1]

to prepare a compound of Formula S-6

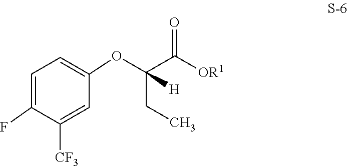

wherein R¹ is C₁-C₆ alkyl; and

treating a compound of Formula S-6 with compound 7

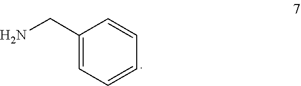

Embodiment B2. The method of Embodiment B1 wherein treating compound R-2 to prepare the compound of Formula R-4 comprises

treating compound R-2 with a chlorinating agent to prepare compound R-8

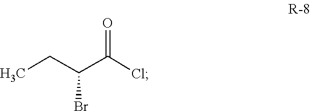

and

treating compound R-8 with a C₁-C₆ alkanol or a salt thereof.

Embodiment B3. The method of Embodiment B2 wherein the chlorinating agent is thionyl chloride.

Embodiment B4. The method of any of Embodiments B1 through B3 wherein R¹ is CH₃.

Embodiment B5. The method Embodiment B wherein compound R-2 is converted to compound S-1 by the method comprising

treating compound R-2 with a chlorinating agent to prepare a compound of Formula R-8

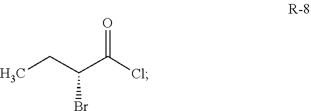

treating compound R-8 with compound 7

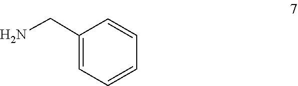

to prepare compound R-9

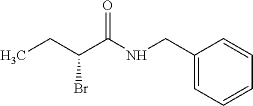

and

treating compound R-9 with compound 5

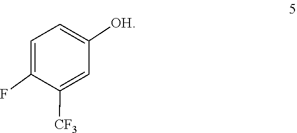 [SMILES: Oc1ccc(F)c(C(F)(F)F)c1]

Embodiment B6. The method of Embodiment B5 wherein the chlorinating agent is thionyl chloride.

Embodiment C1. The method of Embodiment C wherein compound R-2 is converted to compound S-1 by the method comprising

treating compound R-2 with a C₁-C₆ alkanol to prepare a compound of Formula R-4;

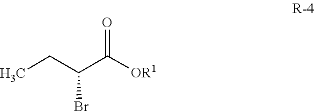

wherein R¹ is C₁-C₆ alkyl;

treating a compound of Formula R-4 with compound 5

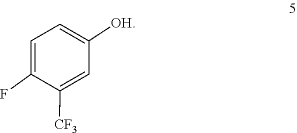 [SMILES: Oc1ccc(F)c(C(F)(F)F)c1]

to prepare a compound of Formula S-6

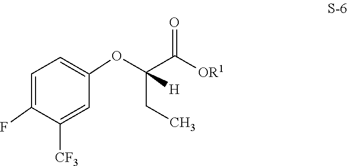

wherein R¹ is C₁-C₆ alkyl; and

treating a compound of Formula S-6 with compound 7

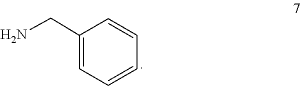

Embodiment C2. The method of Embodiment C1 wherein treating compound R-2 to prepare compound R-4 comprises

treating compound R-2 with a chlorinating agent to prepare compound R-8

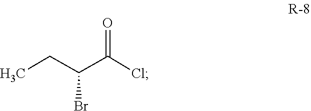

and

treating compound R-8 with a C₁-C₆ alkanol or a salt thereof.

Embodiment C3. The method of Embodiment C2 wherein the chlorinating agent is thionyl chloride.

Embodiment C4. The method of any of Embodiments C1 through C3 wherein R¹ is CH₃.

Embodiment C5. The method Embodiment C wherein compound R-2 is converted to compound S-1 by the method comprising

treating compound R-2 with a chlorinating agent to prepare compound R-8

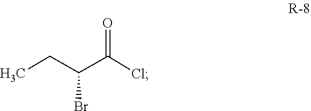

treating compound R-8 with compound 7

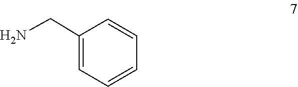

to prepare compound R-9

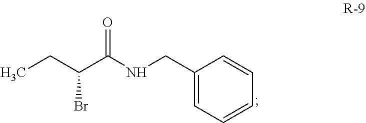

and

treating compound R-9 with compound 5

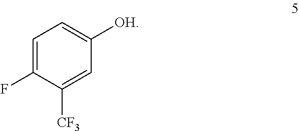 [SMILES: Oc1ccc(F)c(C(F)(F)F)c1]

Embodiment C6. The method of Embodiment C5 wherein the chlorinating agent is thionyl chloride.

Embodiment D1. The method of the Embodiment D wherein the chlorinating agent is thionyl chloride.

Embodiments of this invention, including Embodiments A1 through A6, B1 through B6, C1 through C6 and D1 above as well as any other embodiments described herein, can be combined in any manner, and the descriptions of variables in the embodiments pertain not only to the compounds of Formula S-1 but also to the starting compounds and intermediate compounds of Formulae 2 through 11, useful for preparing the compounds of Formula S-1.

In the following Schemes the definitions of R¹ in the compounds of Formulae R-4 and S-6 below are as defined above in the Summary of the Invention and description of embodiments unless otherwise indicated.

The methods described herein provide an efficient and robust synthesis of the compound of Formula S-1.

Obtaining acids of high enantiomeric purity can be accomplished in several ways, including catalytic asymmetric synthesis, chromatographic resolution, extraction resolution, membrane resolution, enzymatic resolution and diastereomeric salt resolution. Resolution of 2-haloacids using optically active 1-(1-naphthyl)ethylamine has been disclosed (JPS61227549). R-2-halobutanoic acids can also be obtained by treatment of racemic 2-halobutanoic acids with 2-haloacid dehalogenases or haloalkane dehalogenases, which selectively react with the S-halo enantiomer, resulting in R-2-halobutanoic acids in high enantiomeric purity (JPH04325096; JPH02238895).

Alternatively, the desired acid can be obtained from another compound with high enantiomeric purity by functional group interconversion. In this instance, preparation of (R)-2-bromobutanoic acid can be readily achieved by diazotization of (R)-2-aminobutanoic acid in the presence of hydrobromic acid (U.S. Pat. No. 9,145,425; JP2011093869; Bioorg. Med. Chem. Lett. 2008, 18, 732; J. Med. Chem. 2009, 52, 4443; Helv. Chim. Acta 1983, 66, 1028; and J. Org. Chem. 2006, 71, 3332). As summarized in Scheme 1, the compound of Formula S-1 can be prepared from the compound of Formula R-2, wherein the compound of Formula R-2 is obtained by diazotization of the compound of Formula R-3 in the presence of hydrobromic acid. Conversion of the compound of Formula R-2 to the compound of Formula S-1 can be accomplished by any of several reaction sequences subsequently described herein.

The diazotization can be accomplished using an alkali metal nitrite such as sodium nitrite or potassium nitrite. Sodium nitrite is preferred. The reaction can be run in an aqueous mixture, optionally in the presence of an organic solvent such as toluene, usually at about −10 to 10° C. Alternatively, the hydrobromic acid can be generated in situ, such as by a combination of sulfuric acid and sodium bromide or potassium bromide.

Alternatively, the treatment of the compound of Formula R-3 may be conducted under Knoevenagel conditions using an alkyl nitrite such as methyl nitrite, amyl nitrite or tert-butyl nitrite in a mildly acidic solvent system. A mixture of bis(trifluoromethane)-sulfonimide (CF₃SO₂)₂NH, (TFSI-H) and glacial acetic acid can be used as a mild acidic agent.

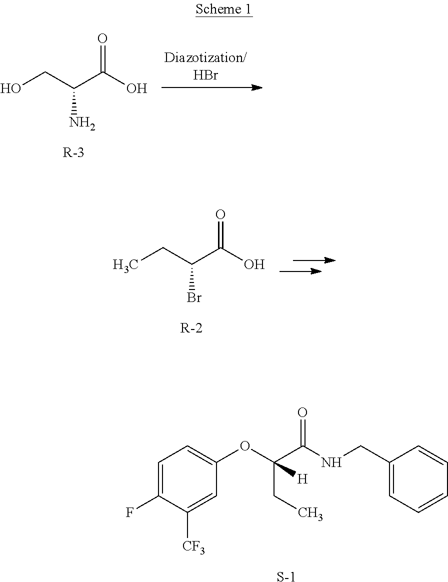

Notably, the configuration at the chiral center is retained. Without being bound by any theory, retention of configuration suggests that anchimeric assistance, or neighboring group participation, plays a role in the reaction as shown in Scheme 2. The carboxylate group in the initially formed diazonium species I-10 can displace dinitrogen by an S_{N}2 attack to provide the α-lactone I-11. Due to ring strain, α-lactone I-11 is readily opened by the nucleophilic bromide ion in a second S_{N}2 step to form the product compound R-2.

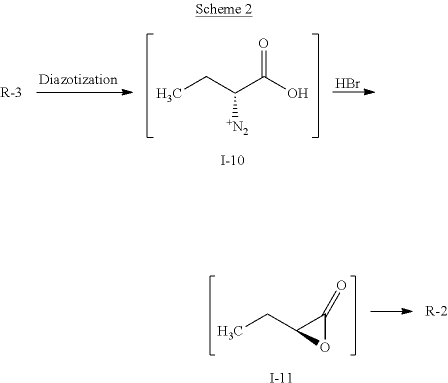

As shown in Scheme 3, compound R-2 can be converted to a compound of Formula R-4 by treatment with a C₁-C₆ alkanol by acid-catalyzed esterification, by dehydration with water-absorbing agents such as zeolites, or by treatment with an acid chloride such as acetyl chloride in the presence of a C₁-C₆ alkanol (Clinica Chimica Acta 1981, 111, 91-98). Preferred are the methyl or ethyl ester, and more preferred is the methyl ester. Alternatively, compound R-2 can be converted to the compound of Formula R-4 by treatment with a chlorinating agent to prepare compound R-8 followed by treatment with a C₁-C₆ alkanol. Suitable chlorinating agents include POCl₃, SOCl₂, (COCl)₂ or COCl₂. Thionyl chloride, SOCl₂, is a preferred chlorinating agent. Suitable solvents include acetonitrile, dichloroethane, toluene, tetrahydrofuran, dimethyl sulfoxide or N,N-dimethylformamide Preferred solvents include N,N-dimethylformamide, dichloroethane, toluene or acetonitrile, more preferably toluene.

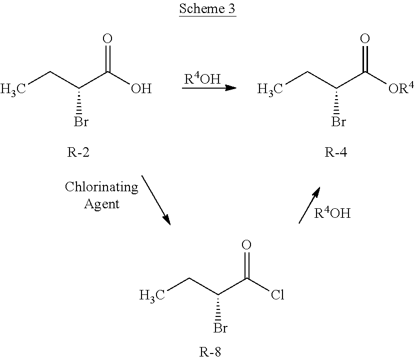

Compounds of Formula R-4 can also be prepared by kinetic resolution of compound rac-4 using lipase enzymes (CN105063120).

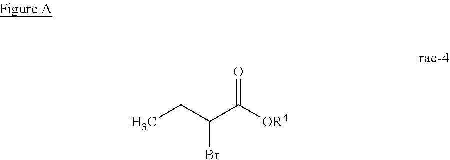

As shown in Scheme 4, the compound of Formula R-4 can be treated with compound 5 in the presence of a base to provide the compound of Formula S-6. (See JP2011093869). Suitable solvents include acetonitrile, dichloroethane, toluene, isopropanol, tetrahydrofuran, dimethyl sulfoxide or N,N-dimethylformamide Preferred solvents include dichloroethane, toluene, acetonitrile or N,N-dimethylformamide, more preferably toluene. Suitable additional bases for the reaction include alkali metal hydrides such as sodium hydride; or alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate; or bases such as lithium bis(trimethylsilyl)amide, sodium bis(trimethylsilyl)amide and lithium diisopropylamide; or tertiary amines such as triethylamine and diisopropylethylamine. Preferred bases include sodium hydroxide, potassium hydroxide, sodium bicarbonate, potassium bicarbonate, sodium carbonate or potassium carbonate, preferably as an aqueous solution.

The compound of Formula S-6 can be treated with compound 7 (i.e. benzylamine) to provide compound S-1. Preferably, the treatment comprises heating the compound of Formula S-6 with about 2 to 5 molar equivalents of compound 7, such as about three equivalents, at about 100 to 125° C., such as about 110 to 120° C. Optionally, a solvent such as toluene can be used. The crude material obtained after removal of excess benzylamine can be recrystallized from a mixture of isopropanol and water to provide compound S-1.

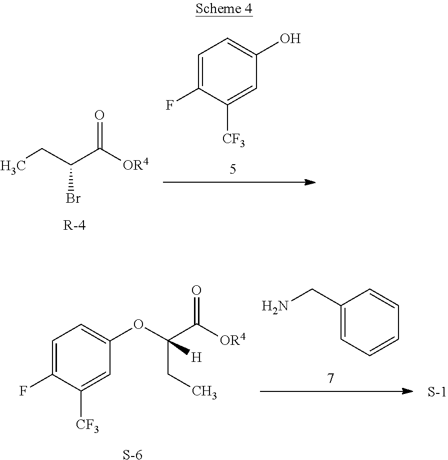

Alternatively, as shown in Scheme 5, compound R-8, prepared as in Scheme 3, can be treated with a compound of Formula 7 in the presence of an additional base to prepare compound R-9. Suitable solvents include acetonitrile, dichloroethane, toluene, tetrahydrofuran, dimethyl sulfoxide or N,N-dimethylformamide Preferred solvents include N,N-dimethylformamide, dichloroethane, toluene or acetonitrile, more preferably toluene. Suitable additional bases for the reaction include alkali metal hydrides such as sodium hydride; or alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate; or bases such as lithium bis(trimethylsilyl)amide, sodium bis(trimethylsilyl)amide and lithium diisopropylamide; or tertiary amines such as triethylamine and diisopropylethylamine. Preferred bases include sodium hydroxide, potassium hydroxide, sodium bicarbonate, potassium bicarbonate, sodium carbonate or potassium carbonate, preferably as an aqueous solution.

Compound R-9 can be treated with compound 5 in the presence of an additional base to prepare compound S-1. Suitable solvents include acetonitrile, dichloroethane, toluene, isopropanol, tetrahydrofuran, dimethyl sulfoxide or N,N-dimethylformamide Preferred solvents include N,N-dimethylformamide, dichloroethane, toluene or acetonitrile, more preferably toluene. Suitable additional bases for the reaction include alkali metal hydrides such as sodium hydride; or alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate; or bases such as lithium bis(trimethylsilyl)amide, sodium bis(trimethylsilyl)amide and lithium diisopropylamide; or tertiary amines such as triethylamine and diisopropylethylamine Preferred bases include sodium hydroxide, potassium hydroxide, sodium bicarbonate, potassium bicarbonate, sodium carbonate or potassium carbonate, preferably as an aqueous solution.

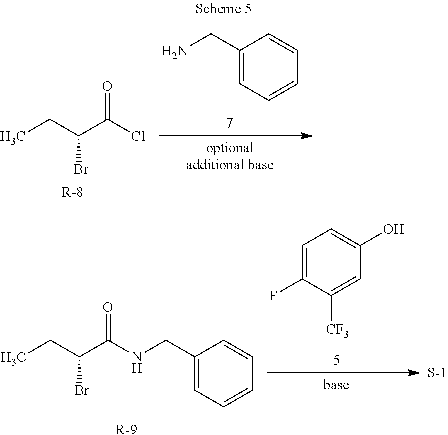

Compound R-9 can also be prepared by kinetic resolution of the compound of Formula rac-9 using haloalkane dehalogenases (Adv. Synth. Catal. 2011, 353, 93-44).

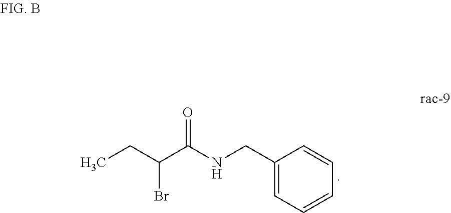

In some embodiments, each of compounds of Formulae R-2, R-4, R-8, R-9 and S-6 can be isolated after preparation and before being carried into the next step. Alternatively, two or more of the steps from the compound of Formula R-2 to the compound of Formula S-1 can be combined without isolating the intermediate compound. For example, if the compound of Formula R-2 is extracted from the aqueous phase with toluene, it can be treated with the chlorinating agent without isolation to prepare the compound of Formula R-8. In other embodiments, conversion of the compound of Formula R-2 to the compound of Formulae R-6 or R-9 can be carried out without isolating the compound of Formula R-8. In another embodiment, the compound of Formula R-8 can be converted to the compound of Formula S-1 without isolating the compound of Formula R-9. In another embodiment, conversion of the compound of Formula R-2 to the compound of Formula S-1 can be accomplished without isolating the compounds of Formulae R-8 and R-9. In another embodiment, conversion of the compound of Formula R-2 to the compound of Formula S-8 can be accomplished without isolating the compounds of Formulae R-8 and R-4. In another embodiment, conversion of the compound of Formula R-2 to the compound of Formula S-1 can be accomplished without isolating the compounds of Formulae R-8, R-4 and S-8.

It is recognized that some reagents and reaction conditions described above for preparing compounds of Formulae 1-11 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formulae 1-11. One skilled in the art will also recognize that it may be necessary to perform a combination of the steps illustrated in the above schemes in an order other than that implied by the particular sequence presented to prepare the compounds of Formulae 1-11. One skilled in the art will also recognize that compounds of Formulae 1-11 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight. The abbreviation “h” stands for “hour” or “hours”. The abbreviation “GCA” stands for “gas chromatographic analysis” and the abbreviation “LCA” stands for “liquid chromatographic analysis.”

### Synthesis Example 1

### Step 1: Preparation of (R)-2-bromobutanoic Acid

To a one-liter round bottomed flask fitted with stirrer, dropping funnel, condenser and thermometer pocket were charged (R)-2-aminobutanoic acid (42.8 g, 0.40 mol) and hydrobromic acid (40% aq., 244.0 g, 1.20 mol). The resulting mixture was cooled to −10 to 0° C. An aqueous solution of sodium nitrite (26.3% aq., 41.4 g, 0.60 mol) was added dropwise to the reaction mixture over a 3-h period while maintaining the reaction mixture at −10 to 0° C. After the addition was complete, the mixture was stirred for one h. The aqueous layer was extracted with toluene (3×240 g). The aqueous layer was acidified with 34% HCl (124.0 g, 1.15 mol) at 25° C. Toluene (660 g) was added and the resulting mixture was stirred for 1 h at −10 to 0° C. The aqueous layer was extracted with toluene (4×230 g) at −10 to 0° C. The combined organic phases were concentrated to dryness at 40 to 50° C. to obtain the title compound (128 g) with purity (LCA) of 91% and yield of 82-85%, ee 96-97%.

### Step 2. Preparation of (R)-2-bromobutanoic Acid Chloride

A three-liter round bottomed flask fitted with stirrer, condenser, thermometer pocket, dropping funnel, nitrogen inlet and scrubber was flushed with nitrogen and charged with a solution of R-2-bromobutanoic acid (210.73 g), i.e. the title compound of Step 1, in toluene (210 g) solution with stirring. The solution was heated to about 48 to 50° C. To this thionyl chloride (126.3 g) was added through the dropping funnel for 1.5 to 2 h at 48 to 50° C. Sulfur dioxide and hydrochloric acid gases evolved from the reaction were scrubbed into a sodium hydroxide aqueous solution. The reaction mass was heated at 60° C. until completion of the reaction, then concentrated under reduced pressure. (R)-2-bromobutanoic acid chloride in toluene solution (439 g) was obtained. Purity by GCA was 99.3%, ee was 95.1% and yield was 99% from (R)-2-bromobutanoic acid.

### Step 3: Preparation of (R)-2-bromo-N-benzylbutanamide

A three-liter round bottomed flask fitted with stirrer, condenser, thermometer pocket, dropping funnel and nitrogen inlet was charged with a solution of (R)-2-bromobutyric acid chloride (443.5 g) in toluene (744 g) with stirring. The solution was cooled to −2 to 3° C. To this solution benzylamine (118.5 g) was added through the dropping funnel for a 1 to 1.5 h period at −2 to 3° C. Sodium hydroxide aqueous solution (440 g) was then added dropwise for a 1-h period at −2 to 3° C. The reaction mass was stirred at −2 to 3° C. until completion of the reaction, then prepared for phase separation. The organic phase was separated. The aqueous phase was extracted with toluene and the organic phases were combined and washed with water. The combined organic phase was evaporated to dryness to provide the title compound (256 g). Purity by GCA was 98.74%, ee was 94% and yield 98.7%.

### Step 4: Preparation of (2S)—N-benzyl-2-(4-fluoro-3-trifluoromethylphenoxy)-butanoic Amide

A three-liter round bottomed flask fitted with stirrer, condenser, thermometer pocket, vacuum outlet and azeotrope water removal setup was charged with 4-fluoro-3-(trifluoromethyl)phenol (253.5 g), sodium hydroxide (100 g) and toluene (500 g) with stirring. The reaction mixture was heated to 55-60° C. and water was removed by azeotropic distillation under reduce pressure. Then, a solution of (R)-2-bromo-N-benzyl butanamide (257 g), i.e. the title compound from Step 3, in toluene (500 g) was added to the reaction mixture at 50-55° C. The reaction mass was heated at 85-100° C. until completion of reaction. The reaction mixture was washed with dilute NaOH solution and the phases were separated. The aqueous phase was extracted with toluene. The combined organic phases were washed with brine solution. The brine-washed organic phase was treated for toluene recovery under reduced pressure until dryness. The resulting crude product was purified in isopropyl and water mixture. The title compound was obtained as a solid (317.51 g) with purity of 99.6%, ee of 98.9% and yield of 88.5%.

What is claimed is:

1. A method for preparing compound S-1 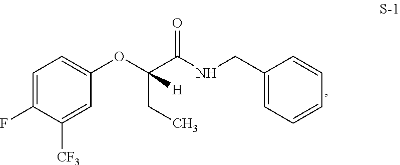 [SMILES: CC[C@H](Oc1ccc(F)c(C(F)(F)F)c1)C(=O)NCc1ccccc1]
from compound R-2 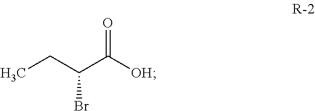
wherein compound R-2 is prepared by
treating compound R-3 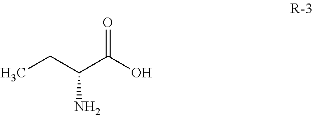
with an alkali metal nitrite and hydrobromic acid.

2. The method of claim 1 wherein compound R-2 is converted to compound S-1 by the method comprising
treating compound R-2 with a C₁-C₆ alkanol to prepare a compound of Formula R-4; 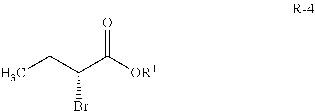
wherein R¹ is C₁-C₆ alkyl;
treating a compound of Formula R-4 with compound 5 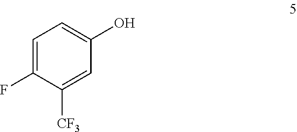 [SMILES: Oc1ccc(F)c(C(F)(F)F)c1]
to prepare a compound of Formula S-6 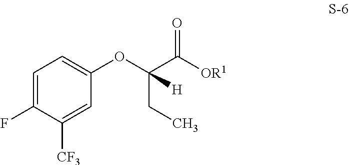
wherein R¹ is C₁-C₆ alkyl; and
treating a compound of Formula S-6 with compound 7

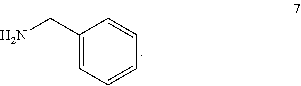

3. The method of claim 2 wherein treating compound R-2 to prepare the compound of Formula R-4 comprises
treating compound R-2 with a chlorinating agent to prepare compound R-8 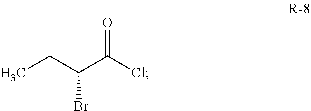
and
treating compound R-8 with a C₁-C₆ alkanol or a salt thereof.

4. The method of claim 3 wherein the chlorinating agent is thionyl chloride.

5. The method of claim 2 wherein R¹ is CH₃.

6. The method of claim 1 wherein compound R-2 is converted to compound S-1 by the method comprising
treating compound R-2 with a chlorinating agent to prepare compound R-8 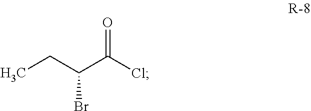
treating compound R-8 with compound 7 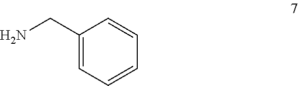
to prepare compound R-9 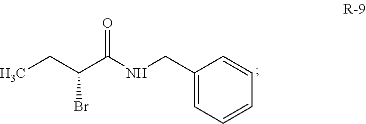
and
treating compound R-9 with compound 5

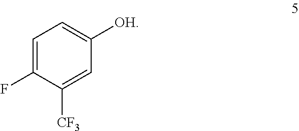 [SMILES: Oc1ccc(F)c(C(F)(F)F)c1]

7. The method of claim 6 wherein the chlorinating agent is thionyl chloride.

8. A method for preparing compound S-1 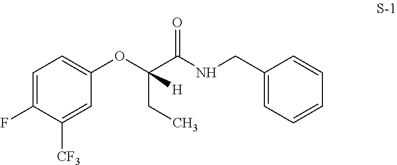 [SMILES: CC[C@H](Oc1ccc(F)c(C(F)(F)F)c1)C(=O)NCc1ccccc1]
from compound R-2 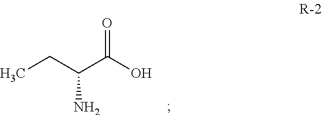
wherein compound R-2 is prepared by
treating compound R-3 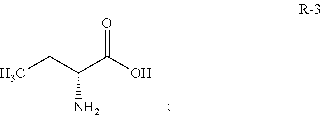
with an alkali metal nitrite and hydrobromic acid;
the method further comprising converting compound R-2 to compound S-1.

9. The method of claim 8 wherein compound R-2 is converted to compound S-1 by the method comprising
treating compound R-2 with a C₁-C₆ alkanol to prepare a compound of Formula R-4; 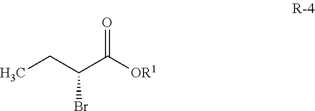
wherein R¹ is C₁-C₆ alkyl;
treating a compound of Formula R-4 with compound 5 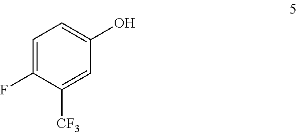 [SMILES: Oc1ccc(F)c(C(F)(F)F)c1]
to prepare a compound of Formula S-6 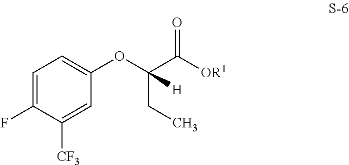
wherein R¹ is C₁-C₆ alkyl; and
treating a compound of Formula S-6 with compound 7

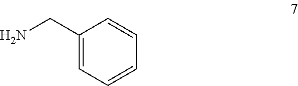

10. The method of claim 9 wherein treating compound R-2 to prepare compound R-4 comprises
treating compound R-2 with a chlorinating agent to prepare compound R-8 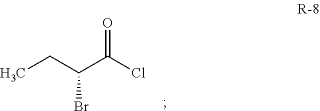
and
treating compound R-8 with a C₁-C₆ alkanol or a salt thereof.

11. The method of claim 10 wherein the chlorinating agent is thionyl chloride.

12. The method of claim 9 wherein R¹ is CH₃.

13. The method of claim 8 wherein compound R-2 is converted to compound S-1 by the method comprising
treating compound R-2 with a chlorinating agent to prepare compound R-8 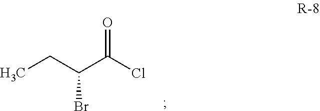
treating compound R-8 with compound 7 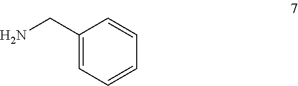
to prepare compound R-9 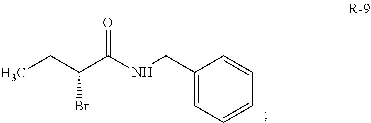
and
treating compound R-9 with compound 5

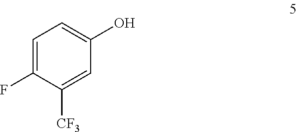 [SMILES: Oc1ccc(F)c(C(F)(F)F)c1]

14. The method of claim 13 wherein the chlorinating agent is thionyl chloride.

15. A method for preparing compound S-1 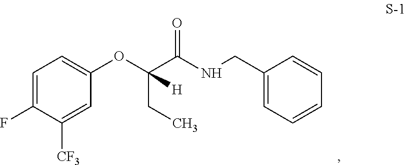 [SMILES: CC[C@H](Oc1ccc(F)c(C(F)(F)F)c1)C(=O)NCc1ccccc1]
the method comprising:
treating compound R-3 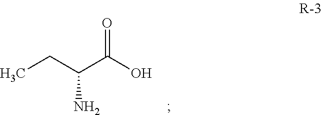
with an alkali metal nitrite and hydrobromic acid to prepare compound R-2 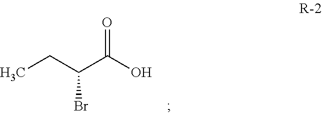
and
converting compound R-2 to the compound of S-1.

16. The method of claim 15 wherein compound R-2 is converted to compound S-1 by the method comprising
treating compound R-2 with a C₁-C₆ alkanol to prepare a compound of Formula R-4; 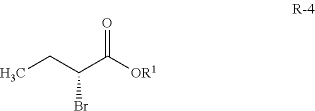
wherein R¹ is C₁-C₆ alkyl;
treating a compound of Formula R-4 with compound 5 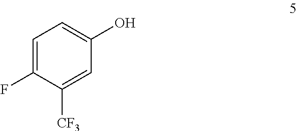 [SMILES: Oc1ccc(F)c(C(F)(F)F)c1]
to prepare a compound of Formula S-6 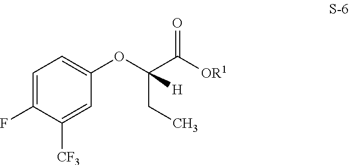
wherein R¹ is C₁-C₆ alkyl; and
treating a compound of Formula S-6 with compound 7

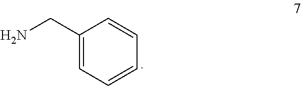

17. The method of claim 16 wherein treating compound R-2 to prepare the compound of Formula R-4 comprises
treating compound R-2 with a chlorinating agent to prepare compound R-8 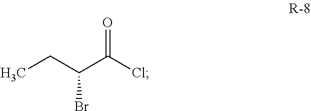
and
treating compound R-8 with a C₁-C₆ alkanol or a salt thereof.

18. The method of claim 17 wherein the chlorinating agent is thionyl chloride.

19. The method of claim 16 wherein R¹ is CH₃.

20. The method of claim 15 wherein compound R-2 is converted to compound S-1 by the method comprising
treating compound R-2 with a chlorinating agent to prepare compound R-8 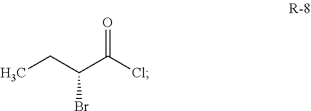
treating compound R-8 with compound 7 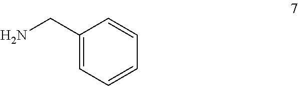
to prepare compound R-9 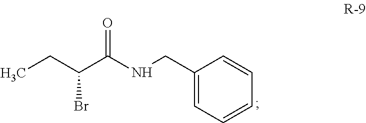
and
treating compound R-9 with compound 5

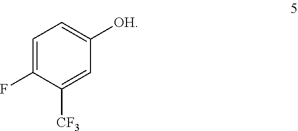 [SMILES: Oc1ccc(F)c(C(F)(F)F)c1]

21. The method of claim 20 wherein the chlorinating agent is thionyl chloride.

22. A method for preparing compound S-1 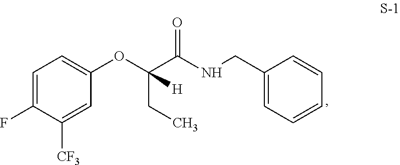 [SMILES: CC[C@H](Oc1ccc(F)c(C(F)(F)F)c1)C(=O)NCc1ccccc1]
the method comprising:
treating compound R-3 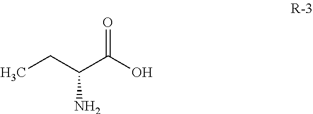
with an alkali metal nitrite and hydrobromic acid to prepare compound R-2 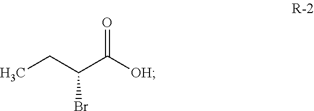
treating compound R-2 with a chlorinating agent to prepare compound R-8 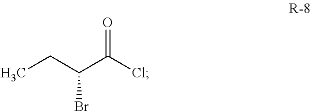
treating compound R-8 with compound 7 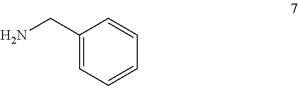
to prepare compound R-9 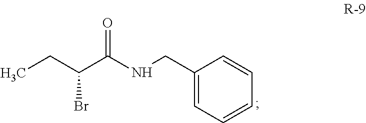
and
treating compound R-9 with compound 5

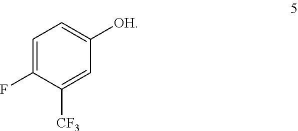 [SMILES: Oc1ccc(F)c(C(F)(F)F)c1]

23. The method of claim 22 wherein the chlorinating agent is thionyl chloride.

24. The method of claim 20 wherein the chlorinating agent is thionyl chloride.
